(19) United States
(12) Patent Application Publication
(10) Pub. No.: US 2016/0051193 A1
(43) Pub. Date: Feb. 25, 2016

(54) APPARATUS FOR AND METHOD OF DETECTING BIOLOGICAL INFORMATION

(71) Applicant: SAMSUNG ELECTRONICS CO., LTD., Suwon-si (KR)

(72) Inventors: Jinyoung PARK, Hwaseong-si (KR); Moonsook LEE, Seoul (KR); Woochang LEE, Anyang-si (KR); Junyoung LEE, Seongnam-si (KR); Seongho CHO, Gwacheon-si (KR); Dongho KIM, Seoul (KR); Jungmok BAE, Seoul (KR)

(73) Assignee: SAMSUNG ELECTRONICS CO., LTD., Suwon-si (KR)

(21) Appl. No.: 14/799,045

(22) Filed: Jul. 14, 2015

(30) Foreign Application Priority Data
Aug. 22, 2014 (KR) 10-2014-0109965

(51) Int. Cl. A61B 5/00; A61B 5/053; A61B 5/117; A61B 5/024

(52) U.S. Cl. CPC A61B 5/681; A61B 5/02438; A61B 5/053; A61B 5/1172; A61B 5/72

(57) ABSTRACT

An apparatus for and method of detecting biological information. The apparatus for detecting biological information includes a supporting member that is attached to an object, a biosignal sensor that is configured to detect biological information of the object and mounted in the supporting member; and a plurality of position sensors configured to detect a position of the apparatus with respect to the object. The position sensors may detect position information of an area of the object where the apparatus is attached. Position data detected by the position sensors may be compared with reference position data.

### CROSS-REFERENCE TO RELATED APPLICATION

This application claims priority from Korean Patent Application No. 10-2014-0109965, filed on Aug. 22, 2014 in the Korean Intellectual Property Office, the disclosure of which is incorporated herein in its entirety by reference.

### BACKGROUND

1. Field

Apparatuses and methods consistent with exemplary embodiments relate to detecting biological information, and more particularly, detecting biological information which include position sensors.

2. Description of the Related Art

As health concerns have increased, various apparatuses for detecting biological information from a subject have been developed. Particularly, as many wearable devices which the subject may directly wear have been widely used, healthcare-specialized devices have been developed.

Recently, different types of optical and electromagnetic sensors are included in such wearable devices. Various information concerning states of living bodies may be obtained via optical sensors or electromagnetic sensors of wearable devices, and the obtained information may be used as indicators of users' health. While the biological information obtained at various times via these sensors included in wearable devices is important, biological information having a known pattern over a predetermined period may be used as reliable indicators of a user's health. If the reproducibility of biological information repeatedly measured is low, the accuracy of the wearable device may be low or a position where the biological information is measured is different from a position where the biological information has been initially measured.

Particularly, when biometric sensors use an optical method, measurement results of the biometric sensors may be greatly influenced by variations in the positions of the biometric sensors in relation to a subject who wears a device including the biometric sensors. Thus, a variety of instrumental or algorithmic technologies have been studied for an accurate analysis of biological information.

### SUMMARY

One or more exemplary embodiments provide apparatuses for measuring biological information of objects, the apparatuses including position sensors.

Still, one or more exemplary embodiments provide methods of detecting biological information of objects.

According to an aspect of an exemplary embodiment, there is provided an apparatus for detecting biological information including a supporting member that is attached to an object; at least one biosignal sensor that is configured to detect biological information of the object and is provided in the supporting member; and a plurality of position sensors configured to detect a position of the apparatus with respect to the object.

The supporting member may be formed of a flexible material.

The position sensors may detect a position of the object in contact with the apparatus based on physical information or biological information obtained from the object.

The biological information may include an impedance, a vascular structure, a bone structure, a surface shape, or a fingerprint of the object.

The position sensors may include impedance sensors or finger scan sensors.

The position sensors may be the impedance sensors provided in the supporting member, and the impedance of the object may be measured a plurality of times by combining the position sensors.

The finger scan sensors may detect an uneven structure or a pattern of a surface of the object by using an optical method, an ultrasonic method, a thermal sensitive method, or a method of sensing a capacitance or electrical conductivity.

The position sensors may be further configured to detect a length of the supporting member or a perimeter of the object.

The supporting member may include a flexible printed circuit board (FPCB).

According to an aspect of another exemplary embodiment, there is provided a method of detecting biological information including activating position sensors of an apparatus for detecting the biological information attached to an object; measuring biological information of the object in a measurement position; and determining an optimum position of the apparatus with respect to the object based on a comparison between the measurement position and a reference position which is stored in the apparatus.

The determining of the optimum position may include: setting the reference position in which other biological information of the object is premeasured; and comparing the biological information measured in the reference position and the biological information measured in the measurement position.

The method may further include determining the measurement position as the optimum position in response to a deviation between the reference position and the measurement position being within a predetermined range.

The method may further include comparing reference position data regarding the reference position to measurement position data regarding the measurement position.

The position sensors may be impedance sensors, and the determining the optimum position may include comparing impedances measured in the reference position and impedances measured in the measurement position.

Reference position data of the reference position and measurement position data of the measurement position may respectively correspond to the impedances measured in the reference position and the measurement position, and corresponding values from among the impedances may be compared with each other to determine the optimum position.

If the measurement position is determined as the optimum position, the biological information of the object may be detected by a biosignal sensor.

The method may further include outputting an instruction to change the measuring position to another measuring position in response to the measurement position being determined as not the optimum position.

The method may further include changing the measurement position in response to the measurement position being determined as not the optimum position.

According to another aspect of an exemplary embodiment, there is provided an apparatus for detecting biological information including: a biosignal sensor that is configured to detect biological information of an object; a plurality of position sensors configured to detect a position of the apparatus with respect to the object; and a processor configured to determine whether the detected information is measured at an optimum position based on the detected position of the apparatus and a prestored reference position.

The processor may be further configured to selectively activate one or more of the plurality of position sensors and the activated one or more of the plurality of position sensors are located to face each other.

### BRIEF DESCRIPTION OF THE DRAWINGS

The above and/or other aspects will be more apparent by describing certain exemplary embodiments, with reference to the accompanying drawings, in which:

FIG. 1 is a view of an apparatus for detecting biological information according to an exemplary embodiment;

FIG. 2 is a view of an example of implementing an apparatus for detecting biological information according to another exemplary embodiment;

FIG. 3 is a flowchart of a method of detecting biological information by using the apparatus for detecting biological information according to exemplary embodiments;

FIG. 4 is a view that illustrates an example of a method of determining a position of the apparatus for detecting biological information;

FIG. 5 is a view that illustrates another example of the method of determining a position of the apparatus for detecting biological information; and

FIG. 6 is a view of a system for detecting biological information, the system including the apparatus for detecting biological information.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail. Expressions such as “at least one of,” when preceding a list of elements, modify the entire list of elements and do not modify the individual elements of the list.

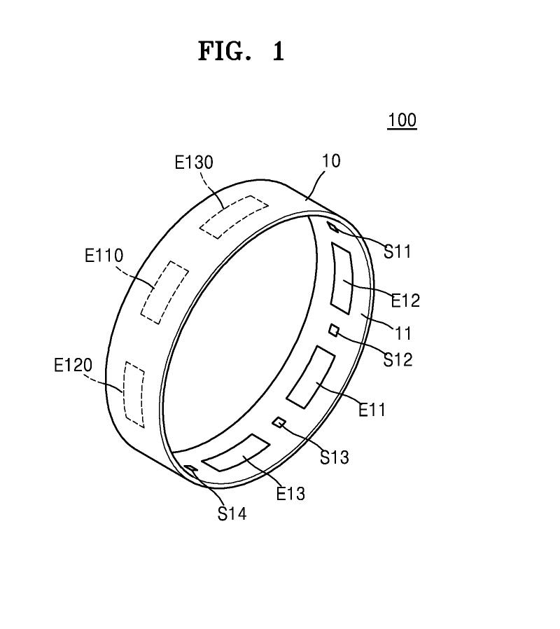

FIG. 1 is a view of an apparatus 100 for detecting biological information according to an exemplary embodiment.

Referring to FIG. 1, the apparatus 100 may include a supporting member 10, which may be attached to an object, one or more biosignal sensors S11, S12, S13, and S14 for detecting biological information of the object and which are disposed or mounted in/on the supporting member 10, and position sensors E11, E12, E13, E110, E120, and E130 for detecting a position of the apparatus 100 with respect to the object.

The supporting member 10 may have an inner side 11 thereof attached to one part of the object and may be formed of a flexible material. The supporting member 10 may have a circular or oval shape so as to surround a portion of the object. When the apparatus 100 is attached to the object, position information with respect to the part of the object where the apparatus 100 is attached to may be obtained by the position sensors E11, E12, E13, E110, E120, and E130, and biological information of the object at that position may be detected by the biosignal sensors S11, S12, S13 or S14. The object may be a part of a living body such as a wrist, ankle, finger, toe, neck, or torso, and the supporting member 10 may be attached to surround the object. Although the supporting member 10 does not completely surround the object, the supporting member 10 may be stably fixed on a surface of the object. The supporting member 10 may include a flexible printed circuit board (FPCB).

The biosignal sensors S11, S12, S13 and S14 may measure the biological information of the object. The biological information may be various kinds of information such as a pulse wave, blood sugar level, heartbeat, breath, stress, or calorie consumption. The biosignal sensors S11, S12, S13 and S14 for detecting biological information may include, for example, sensors for measuring pulse waves. In this case, the sensors for measuring pulse waves may include light-emitting portions including light-emitting diodes (LEDs) or laser diodes, and light-receiving portions including photo diodes, photo transistors (PTrs) or charge-couple devices (CCDs).

At least one of the position sensors E11, E12, E13, E110, E120, and E130 detect a position of the apparatus 100 according to the present exemplary embodiment or a position of the object in contact with the inner side 11 of the supporting member 10 of the apparatus 100. The position may be detected from position information which is obtained from the object by at least one of the position sensors E11, E12, E13, E110, E120, and E130. The information obtained from the object may be physical information such as a size, width, or diameter of the object, or biological information of the object. The biological information which is obtained from the object by the position sensors E11, E12, E13, E110, E120, and E130 may have small variations according to time and may have relatively constant values. For example, the biological information of the object measured by the position sensors E11, E12, E13, E110, E120, and E130 may be an impedance, a vascular structure, a bone structure, a surface shape, or a fingerprint of the object. Thus, the position sensors E11, E12, E13, E110, E120, and E130 may include impedance sensors or finger scan sensors.

The impedance sensors may measure impedances in a local area of the object, and the impedances may vary with the state of skin, fat, muscle, and moisture in the object. In general, an amount of muscle and fat of the object may remain substantially constant as long as the object is not sick or under a severe diet. Depending on positions of the object where impedances are measured, a path of measuring the impedances may differ and the impedances may vary. The impedance corresponding to a body composition in the local area of the object may be relatively constant unless the positions where the impedance is measured change. As a result, the impedances according to the position of the object as measured by the impedance sensors may be considered as denoting the positions of the impedance sensors with respect to the object. Accordingly, if the impedance in the local area of the object is measured and compared with a previously measured impedance, whether a position of the apparatus 100 which includes the impedance sensors has changed compared to an initial position may be determined.

The finger scan sensors may be general sensors for scanning each person's fingerprint. However, the finger scan sensors may also be sensors for scanning a surface shape which appears on the surface of the object, for example, an uneven structure on the surface of the object, or sensors for scanning a surface pattern of the object. The finger scan sensors may use an optical method, an ultrasonic method, a thermal sensitive method, a method of sensing a capacitance or electrical conductivity, or other various methods.

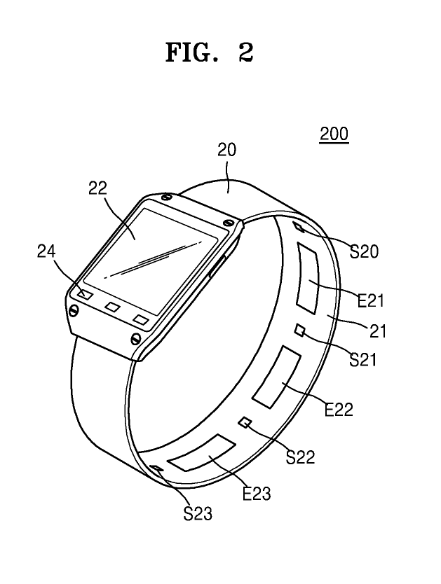

FIG. 2 is a view of an example of implementing an apparatus 200 for detecting biological information according to another exemplary embodiment.

Referring to FIG. 2, the apparatus 200 may include a supporting member 20 which may be attached to or placed on an object, and multiple biosignal sensors S20, S21, S22 and S23 for detecting biological information and position sensors E21, E22, E23, and E23, which are all included in the supporting member 20. The apparatus 200 may further include a display 22 for visually displaying information obtained by the multiple biosignal sensors S20, S21, S22 and S23 for detecting biological information and position sensors E21, E22, E23, and E23, and an input unit 24 for selectively controlling the biosignal sensors S20, S21, S22 and S23 and the position sensors E21, E22, E23, and E23. In addition, the input unit 24 may be connected to a data processor.

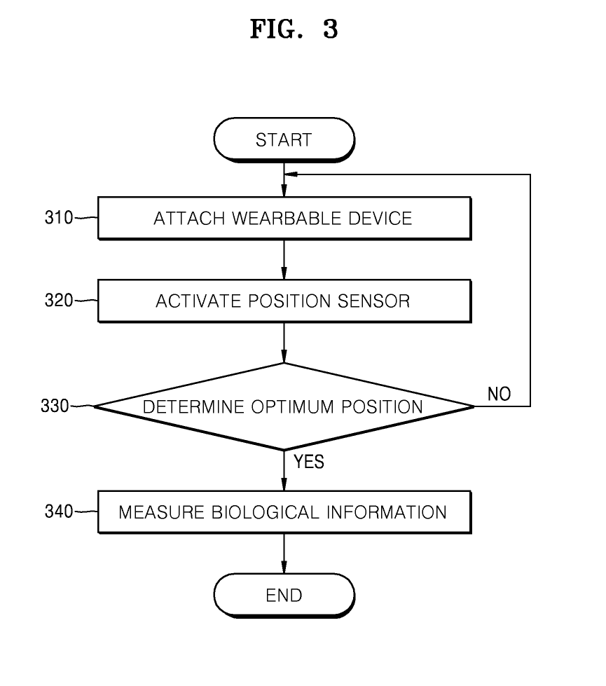

Hereinafter, a method of detecting biological information according to an exemplary embodiment will be described with reference to the drawings. FIG. 3 is a flowchart of the method of detecting biological information by using the apparatus for detecting biological information according to the exemplary embodiments.

Referring to FIG. 3, the apparatus for detecting biological information according to the exemplary embodiments is attached to or placed on one part of an object (operation S310). The apparatus may be a wearable device. The apparatus may be attached to a wrist or an ankle, but the exemplary embodiments are not limited thereto. Next, position sensors of the apparatus are activated (operation S320). The position sensors may detect an impedance in a particular area of the object where the apparatus is attached or a surface shape of the object.

Next, whether the area of the object where the apparatus is attached corresponds to an optimum position is determined by using position information measured by the position sensors in operation S320 (operation S330). The optimum position may be a position where a deviation in a reference position and a measurement position is within a predetermined range. The apparatus for detecting biological information according to the exemplary embodiments may previously measure biological information of the object. A position where the biological information is measured may be the reference position, and position data measured in the reference position may be reference position data. That is, the reference position may be predetermined by detecting the biological information of the object by the apparatus according to the exemplary embodiments. For example, the apparatus may detect the biological information of the object such as pulse waves in area A1 of the object. The area A1 is assumed to be the reference position. The biological information of the object may be measured again in area A2 of the object to check if there is any change in the biological information of the object. The area A2 is assumed to be the measurement position. The reference position A1 and the measurement position A2 may be supposed to be the same or very similar to each other in order to see if there is any change in the biological information of the object. If the deviation between the reference position A1 and the measurement position A2 is within the predetermined range, the measurement position A2 may be determined as being the optimum position.

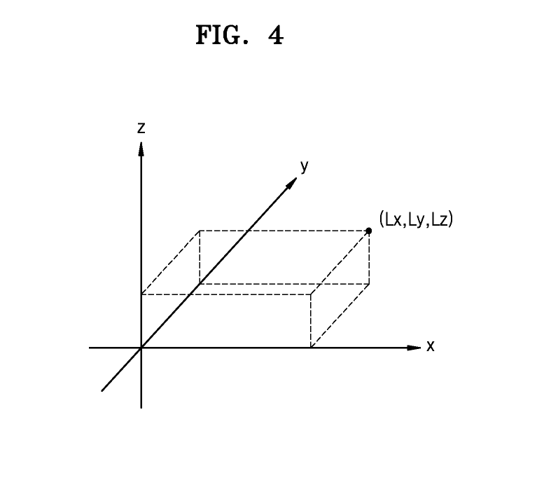

A method of measuring the position of the apparatus for detecting biological information will be described with reference to FIGS. 1, 4, and 5. FIG. 4 illustrates determining a position of the apparatus for detecting biological information according to the exemplary embodiment illustrated in FIG. 1.

Referring to FIGS. 1 and 4, the supporting member 10 has the inner side 11 attached to the object, and the position sensors E11, E12, E13, E110, E120, and E130 included in the supporting member 10 measure impedances from an area in contact with the part of the object. One or more of the position sensors E11, E12, E13, E110, E120, and E130 which measure impedances may be selected and a combination of the selected position sensors may be determined. When previously measured reference position data exists, the position sensors E11, E12, E13, E110, E120, and E130 may be combined in the same way as when the reference position data was measured.

For example, the position sensors E11, E12, E13, E110, E120, and E130 may be combined as follows: position sensors E11 and E110, position sensors E12 and E120, and position sensors E13 and E130, and impedances for each of the combinations may be measured. A plurality of the impedances of the object may be measured by using two, four or more electrodes. Assuming that the measured impedance when the position sensors E11 and E110 are used is Lx1, the measured impedance when the position sensors E12 and E120 are used is Ly1, and the measured impedance when the position sensors E13 and E130 are used is Lz1, and the impedances Lx1, Ly1, and Lz1 may be set up as one piece of position information and displayed as one vector in a three-dimensional graph as shown in FIG. 4. The impedances Lx1, Ly1, and Lz1 may be respectively compared with reference data. If the reference data are Lx0, Ly0, and Lz0, deviations between the reference impedances Lx0, Ly0, and Lz0 and the measured impedances Lx1, Ly1, and Lz1 may be respectively obtained by equation 1 below.

ΔLx=|Lx1−Lx0|

ΔLy=|Ly1−Ly0|

ΔLz=|Lz1−Lz0| [Equation 1]

If each of the deviations ΔLx, ΔLy, and ΔLz is compared with each of the reference impedances Lx0, Ly0, and Lz0 and is within a predetermined range, each of the impedances Lx1, Ly1, and Lz1 may be determined as corresponding to an optimum position range and thus the next operation may proceed. The predetermined range may be 1%, 5%, 10%, or the like with respect to the reference impedances Lx0, Ly0, and Lz0. For example, if each of the deviations ΔLx, ΔLy, and ΔLz is within 5% or 10% from the reference impedances Lx0, Ly0 and Lz0, respectively, each of the impedances Lx1, Ly1, and Lz1 may be determined as being within an optimum position range. In some exemplary embodiments, a sum of the deviations ΔLx+ΔLy+ΔLz is compared with a sum of the reference impedances Lx0+Ly0+Lz0 to determine if each of the deviations ΔLx, ΔLy, and ΔLz is within a predetermined range, and in the affirmative, it may be determined that each of the measured impedances Lx1, Ly1, and Lz1 is within an optimum position range and thus the next operation may proceed. Also, aLx1, bLy1 and cLz1 may be obtained by respectively assigning weight factors a, b and c to the impedances Lx1, Ly1, and Lz1, and thus may be compared with the reference impedances Lx0, Ly0, and Lz0, respectively.

As described above, the position sensors E11, E12, E13, E110, E120, and E130 may be impedance sensors which may measure the impedances from an area in contact with the part of the object, however, the position sensors E11, E12, E13, E110, E120, and E130 are not limited thereto. For example, the position sensors E11, E12, E13, E110, E120, and E130 may be finger scan sensors. The finger scan sensors may detect an uneven structure or a pattern on the surface of the object by using an optical method, an ultrasonic method, a thermal sensitive method, a method of sensing capacitance or electrical conductivity, or the like.

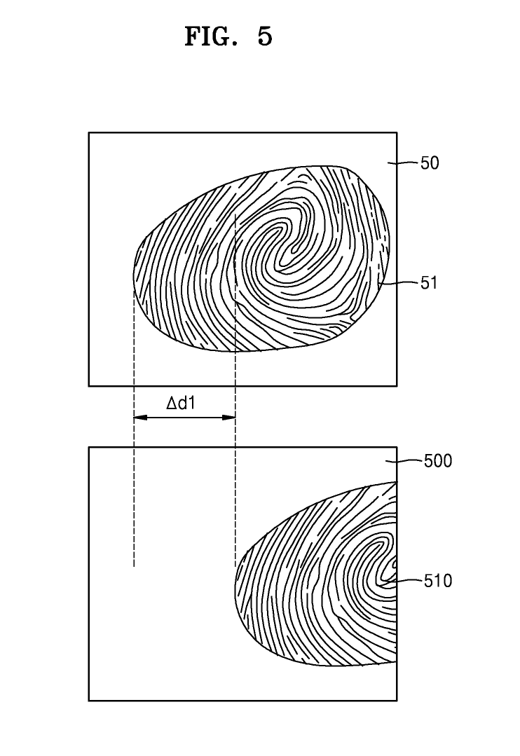

FIG. 5 illustrates a surface shape of the object detected by the finger scan sensors. Referring to FIG. 5, a measurement image 500 of the surface shape of the object as detected by at least one of the position sensors E11, E12, E13, E110, E120, and E130 is compared with a reference image 50 displaying a previously measured surface shape. When the reference image 50 and the measurement image 500 are compared, positions of the same or similar portions of fingerprints may be compared. That is, a deviation between positions of a fingerprint 51 in the reference image 50 and a fingerprint 510 in the measurement image 500 may be denoted as Δd1, and if the position deviation Δd1 is within a predetermined range, the position of the fingerprint 510 in the measurement image 500 may be determined as an optimum position and the next operation may proceed.

Referring to FIG. 3 again, when the position data detected by at least one of the position sensors E11, E12, E13, E110, E120, and E130 is compared with the reference position data and thus it is determined the detected position data is within the predetermined error margin, the biological information of the object may be detected by at least one of the biosignal sensors S20, S21, S22, and S23 (operation S340). The biosignal sensors S20, S21, S22, and S23 may detect various kinds of biological information. The detected biological information may be output together with the position information data detected by at least one of the position sensors E11, E12, E13, E110, E120, and E130.

When the position data detected by at least one of the position sensors E11, E12, E13, E110, E120, and E130 is compared with the reference position data and it is determined that the detected position data is not within the predetermined error margin, a user may be notified of the determination and the position of the apparatus for detecting biological information may be changed. Accordingly, detecting biological information may be started again from attaching the apparatus for detecting biological information to the object. The apparatus for detecting biological information according to the exemplary embodiments may further include units for detecting a length of the supporting member 10 or a perimeter of the object as position sensors. Thus, when the position data regarding the area of the object where the apparatus for detecting biological information is attached is detected, the length of the supporting member 10 or the perimeter of the object may be detected. For example, when the apparatus for detecting biological information is attached to the object, if perimeters of the object are different in the attached position with respect to reference values, the position may be determined as not being an optimum position.

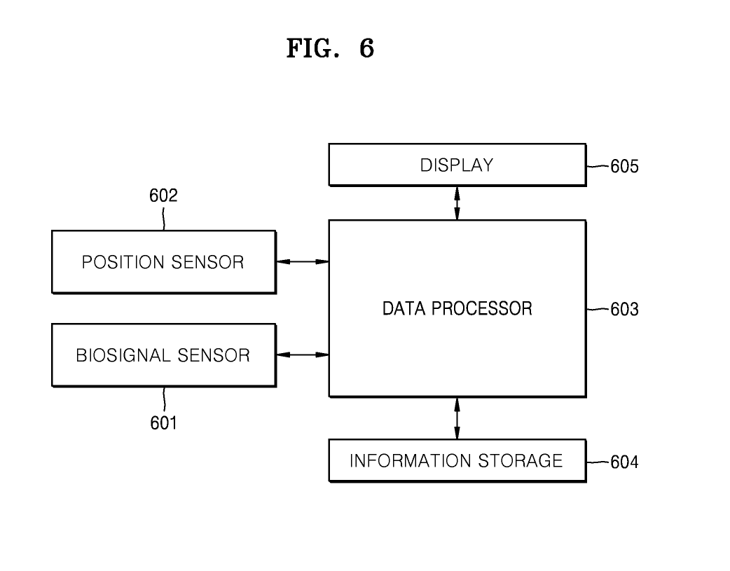

FIG. 6 is a view of a system for detecting biological information, the system including the apparatus for detecting biological information according to one or more exemplary embodiments.

Referring to FIG. 6, the system for detecting biological information may be a wearable device including a biosignal sensor 601 and a position sensor 602. The system for detecting biological information may include a data processor 603 for processing data measured by the biosignal sensor 602 or the position sensor 603, and an information storage 604 for storing data. The data processor may compare the data measured by the biosignal sensor 601 or the position sensor 602 with reference position data and biological information regarding a reference position of an object stored in the information storage 604. The data processor 603 may also control the position sensor 602 or the biosignal sensor 601 so as to detect position information or biological information. The system for detecting biological information may further include a display 605 for displaying predetermined information. The display 605 may output the biological information measured by the biosignal sensor 601 along with the position data, thereby allowing a user to recognize the biological information and the position data. The user may evaluate a health state of the object by using the biological information and position information and may reset the reference position and the reference position data as desired.

As described above, according to one or more of the above exemplary embodiments, when biological information of an object is detected, the biological information measured by comparing a position of an apparatus for detecting biological information attached to the object with a reference position is compared with a reference biological information. Thus, the utility of the detected biological information may be improved.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for detecting biological information, the apparatus comprising:
a supporting member that is configured to attach to an object;
at least one biosignal sensor that is configured to detect biological information of the object and is provided in the supporting member; and
a plurality of position sensors that are configured to detect a position of the apparatus with respect to the object.

2. The apparatus of claim 1, wherein the supporting member is formed of a flexible material.

3. The apparatus of claim 1, wherein the position sensors are further configured to detect a position of the object in contact with the apparatus based on physical information or biological information obtained from the object.

4. The apparatus of claim 3, wherein the biological information comprises an impedance, a vascular structure, a bone structure, a surface shape, or a fingerprint of the object.

5. The apparatus of claim 4, wherein the plurality of position sensors comprise impedance sensors or finger scan sensors.

6. The apparatus of claim 5, wherein the plurality of position sensors comprise the impedance sensors provided in the supporting member, and
the impedance of the object is measured a plurality of times by combining the impedance sensors.

7. The apparatus of claim 5, wherein the position sensors comprise the finger scan sensors, and the finger scan sensors are configured to detect an uneven structure or a pattern of a surface of the object by using an optical method, an ultrasonic method, a thermal sensitive method, or a method of sensing a capacitance or electrical conductivity.

8. The apparatus of claim 1, wherein the position sensors are further configured to detect a length of the supporting member or a perimeter of the object.

9. The apparatus of claim 1, wherein the supporting member comprises a flexible printed circuit board.

10. A method of detecting biological information, the method comprising:
activating position sensors of an apparatus attached to an object;
measuring biological information of the object in a measurement position; and
determining an optimum position of the apparatus with respect to the object based on a comparison between the measurement position and a reference position which is stored in the apparatus.

11. The method of claim 10, wherein the determining the optimum position comprises:
setting the reference position in which other biological information of the object is premeasured;
comparing the biological information measured in the reference position and the biological information measured in the measurement position.

12. The method of claim 11, further comprising determining the measurement position as the optimum position in response to a deviation between the reference position and the measurement position being within a predetermined range.

13. The method of claim 11, further comprising comparing reference position data regarding the reference position to measurement position data regarding the measurement position.

14. The method of claim 11, wherein the position sensors comprise impedance sensors, and the determining the optimum position comprises comparing impedances measured in the reference position and impedances measured in the measurement position.

15. The method of claim 14, wherein reference position data of the reference position and measurement position data of the measurement position respectively correspond to the impedances measured in the reference position and the measurement position, and corresponding values from the impedances are compared with each other to determine the optimum position.

16. The method of claim 11, wherein in response to the measurement position being determined as the optimum position, the biological information of the object is detected by a biosignal sensor.

17. The method of claim 11, further comprising outputting an instruction to change the measuring position to another measuring position in response to the measurement position being determined as being the optimum position.

18. The method of claim 11, further comprising changing the measurement position in response to the measurement position being determined as not the optimum position.

19. An apparatus for detecting biological information, comprising:
a biosignal sensor that is configured to detect biological information of an object;
a plurality of position sensors configured to detect a position of the apparatus with respect to the object; and
a processor configured to determine whether the detected biological information is measured at an optimum position based on the detected position of the apparatus and a prestored reference position.

20. The apparatus of claim 19, wherein the processor is further configured to selectively activate one or more of the plurality of position sensors and the activated one or more of the plurality of position sensors are located to face each other.
